(19) United States
(12) Patent Application Publication
(10) Pub. No.: US 2003/0093043 A1
(43) Pub. Date: May 15, 2003

(54) Multi-functional absorption pad and methods for making the same

(76) Inventors: Taeyong Yi, Blue Bell, PA (US); Jongtaek Lee, Daegu (KR); Taesung Lee, Daegu (KR)

Correspondence Address: Johney U. Han, Morrison & Foerster LLP, 755 Page Mill Road, Palo Alto, CA, 94304-1018, US

(21) Appl. No.: 10/120,601

(22) Filed: Apr. 10, 2002

Related U.S. Application Data
(63) Continuation-in-part of application No. PCT/KR01/0079, filed on May 4, 2001.

(30) Foreign Application Priority Data
Apr. 11, 2001 (KR) 2001-19150

(51) Int. Cl. A61F 13/15; A61F 13/20

(52) U.S. Cl. 604/359; 604/360; 604/367; 604/374; 604/378

(57) ABSTRACT

A multi-functional absorption pad and methods for making the same are disclosed herein. The absorption pad consists of a menstrual pad which may be used to absorb bodily fluids, such as menstrual blood and urine, while simultaneously alleviating the effects of dysmenorrhea, reducing malodorous scents, and having an antibacterial effect. The absorption pad contains a ceramic substance disposed within an absorbent core which helps to absorb the bodily fluids. The ceramic or oxidic substance is formed of a mixture of fine oxide particles, which include SiO₂ (45-90% weight), Al₂O₃ (0.5-30.1% weight), Fe₂O₃ (0.5-30.1% weight), Fe₃O₄ (0.5-30.1% weight), and TiO₂ (0-10% weight). The fine oxide particles are processed in different forms including granular, pellet, and powder form.

### CROSS-REFERENCE TO RELATED APPLICATIONS

This application is a continuation-in-part of PCT/KR01/00729, filed May 4, 2001, designating the U.S., and also claims the benefits of foreign priority under 35 U.S.C. §119 of Korean Patent Application Serial No. 2001-19150 filed on Apr. 11, 2001 in the Korean Intellectual Property Office.

### TECHNICAL FIELD OF THE INVENTION

The present invention relates to a pad for absorbing bodily fluids, the pad comprising biocompatible ceramic substances. More particularly, it relates to a multi-functional menstrual pad comprising metal oxide, semi-metal oxide, or ceramic containing substances and methods for making the same. The pad being able to relieve cramping in women, e.g., dysmenorrhea, while also simultaneously mitigating malodorous scents and bacterial growth. The pad being comprised of a mixture of fine oxide particles, which may include SiO₂ (45-90% weight), Al₂O₃ (0.5-30.1% weight), Fe₂O₃ (0.5-30.1% weight), Fe₃O₄ (0.5-30.1% weight), and TiO₂ (0.0-10% weight),

### BACKGROUND ART OF THE INVENTION

Dysmenorrhea (i.e., cramping pain associated with menstruation) in women typically results in painful menstruation and has usually been treated by gynecologists. According to statistics, many women (about 60-90%) experience dysmenorrhea on a monthly basis. This in turn, in countries such as the Republic of Korea, has resulted in a yearly shortage of female labor productivity of about 6-10 million hours and in countries such as the United States, lost female productivity of about 140 million hours per year.

There are two types of dysmenorrhea conditions: (1) primary dysmenorrhea, where the pain usually begins just before or at the onset of menstruation, and (2) secondary dysmenorrhea, which is usually accompanied by lesions in the uterus and where the pain is similar to that of the primary form but usually begins some years after menarche. Since secondary dysmenorrhea is usually accompanied by lesions, woman with this condition should normally receive medical treatment by a gynecologist.

Primary dysmenorrhea is accompanied by cramping that periodically occurs in most women every month and is typically generated in the vicinity of the hypogastrium and upper pubis. Pain associated with primary dysmenorrhea may be transmitted to the lower abdominal region. A few women undergo extreme pain. Statistically, about 50% of women also undergo a plurality of other constitutional symptoms (e.g., lower backache 60%, vomiting 89%, fatigue 85%, dizziness and diarrhea 60%, anorexia and headache 45%, oversensitiveness, a sense of unease, etc.) along with the cramping pain. Additionally, various bacteria, i.e., over 20 different strains, such as colon bacillus, pseudomonas, trichomonas, etc., typically grow in most women's bodies and may be associated with unpleasant odors.

Primary dysmenorrhea usually occurs before menstrual cramping pain or it occurs within a few hours on the same day generating the menstrual cramp pain, continues for one or two days, and typically does not exceed three days. A large quantity of prostagladins secreted in the menstrual blood usually causes irregular and abnormal uterine contraction as well as an irregular and abnormal imbalance of the uterus. The irregular and abnormal uterine contraction/imbalance ceases oxygenation of the muscle tissue inside the uterus and causes ischemia. It is well known in the art that the irregular and abnormal uterine contraction/imbalance and the ischmia brings about primary dysmenorrhea.

To overcome dysmenorrhea and the symptoms accompanying it, different methods of warming body temperature, e.g., hot baths, have been typically used. Conventional menstrual pads have a simple sanitary function when dealing with menstrual blood during menstruation and cannot reduce dysmenorrhea.

Other conventional methods for reducing dysmenorrhea have been used and are disclosed in the following.

One conventional medical treatment employs medications, e.g., NSAID (Non-Steroidal Anti-Inflammatory Drugs), taken in accordance with medical prescriptions given by a physician. Another treatment includes thermal care by warming the hypogastrium, in particular the area of the stomach. For thermal care, a heating pad or bathing in hot water has been mainly used. Other treatments include jogging or aerobic exercises for warming up the whole female body prior to the menstrual period or during the menstrual period; however, this requires accurate knowledge of when the menstrual period will begin.

Additionally, some women have used oriental medicine, acupuncture, ingesting fish oil (i.e., omega-3 fatty acids), or drinking oriental medicinal teas. Some women also wear hard magnetic materials on their waist or insert products manufactured to emit heat into the vagina.

However, the aforementioned methods have associated disadvantages. For instance, conventional medical treatments may be accompanied by harmful secondary effects such as gastrointestinal injury, dizziness and the like. Other treatments may have a considerable theoretical basis in light of excessive muscle contractions, but these treatments are time consuming, expensive, and are inconvenient to the user. Other methods are also problematic in that they are also time consuming and expensive.

Also in dealing with malodorous scents and bacterial breeding on menstrual pads, various materials have been used, e.g., charcoal, mugwort, poroligenous liquor, and aromatics. But, they may cause color variations and mislead the user in believing in fallacious curative effects. As a result, the treatment methods using them is not employed on a commercial scale.

### SUMMARY OF THE INVENTION

Accordingly, the present invention is directed in part to a multi-functional absorption pad which may be used to absorb bodily fluids and provide relief from the effects of dysmenorrhea and cramping. In particular, a preferable embodiment may include a menstrual pad comprising in part ceramic substances and methods for making the same that substantially obviate one or more of the problems due to limitations and disadvantages of the prior art.

The present invention provides a multi-functional menstrual pad having an additive of metal oxides, semi-metal oxides, or ceramic materials, perhaps a biocompatible ceramic (bioceramic) substances implanted therein which may simultaneously alleviate the pain associated with dysmenorrhea as well as remove malodorous scents and bacterial growth, thereby ensuring a woman's normal social activity even in her condition of dysmenorrhea. The additive or bioceramics comprise a mixture of fine oxide particles, i.e., SiO₂ (45-90% weight), Al₂O₃ (0.5-30.1% weight), Fe₂O₃ (0.5-30.1% weight), Fe₃O₄ (0.5-30.1% weight), and optionally TiO₂ (0-10% weight). These oxide particles may be included as the main components of a clay mineral mixture to form a fine ceramics mixture.

It is another object of the present invention to provide methods for making a multi-functional menstrual pad having in part the bioceramic substances.

To achieve these and other advantages in accordance with the present invention, a variation of such a multi-functional pad may have an absorption surface; an absorbent core preferably positioned under the absorption surface for absorbing bodily fluids, e.g., menstrual blood and urine; a backsheet for supporting the absorbent core; a layer of pulp fluff within the absorbent core; an absorption substance sheet also within the absorbent core and made of an absorption substance. The multi-functional pad preferably has the bioceramic substances dispersed within the pulp fluff, but the bioceramic substance may be placed almost anywhere within the pad so long as it is able to come into contact with the bodily fluids. For such an application, the amount of the bioceramic substance which is effective in relieving pain and mitigating the bacterial growth may range anywhere from 0.2 to 1 gram. The pad may also be configured for various applications. For instance, a pad having the bioceramic substance may be made into a panty-liner type pad, e.g., one that is extra thin, or it may also be configured into a tampon. Additionally, the pad may simply be configured and used as gauze, e.g., medical gauze. The bioceramic substance may also be directly incorporated into an undergarment, e.g., underwear or panties, by any number of conventional methods, e.g., soaking, spraying, and adhering with adhesive.

Aside from the use in a pad, the additive or bioceramic substance may be encapsulated within a package having semi-permeable walls, such as a tea bag-type package. Such an application would contain the additive or bioceramic substance within the package while allowing fluids to be interchanged through the walls. The package may also have an adhesive on a first side; this would allow the package to be adhered to a surface, e.g., an undergarment or panty, while exposing a second side for placement against the skin of a user. After use, the package may then be selectively removable and disposed.

Additional features and advantages of the invention will be set forth in the description which follows, and in part will be apparent from the description, drawings, and claims or may be learned by practice of the invention. It is to be understood that both the foregoing general description and the following detailed description are exemplary and explanatory and are intended to provide further explanation of the invention as claimed.

### BRIEF DESCRIPTION OF THE DRAWINGS

The preferred embodiment of the present invention will be described more specifically with reference to the attached drawings, wherein:

FIG. 1 schematically depicts an exploded perspective view of the general structure of a menstrual pad.

FIG. 2a is a cross-sectional view of the general structure of a menstrual pad which may embody, at least in part, the present invention.

FIG. 2b is an isometric view of a variation on a tea bag-type package containing the bioceramic substances.

FIG. 3 is a concentration curve of measured gas over an elapsed period of time.

FIG. 4a depicts a measurement of the emission power of the bioceramic substances as measured by far infrared rays.

FIG. 4b depicts the emissivity of the bioceramic substances as measured by far infrared rays.

FIG. 5 illustrates the results of an anti-fungal experiment using some of the bioceramic substances.

FIGS. 6a and 6b illustrate the results of an anti-bacterial experiment using some of the bioceramic substance.

FIGS. 7a and 7b illustrate the results of another anti-bacterial experiment using some of the bioceramic substance.

### DETAILED DESCRIPTION OF THE INVENTION

Reference will now be made in detail to the preferred embodiments of the present invention, examples of which are illustrated in the accompanying drawings.

The additive or biocompatible ceramic (bioceramic) substance is comprised of a mixture of fine oxide particles including SiO₂, Al₂O₃, Fe₂O₃, Fe₃O₄, and optionally TiO₂. This mixture may be utilized alone or it may also be used as a main component of a clay mineral mixture to form a fine ceramics mixture.

Among the fine oxide particles, silica (SiO₂) is preferably present in an amount between about 45-90% weight. If the SiO₂ percentage falls outside this range, the dysmenorrhea alleviation effect may be mitigated. The malodorous removal and antibacterial effect, however, may not be mitigated as desirably.

In addition, Al₂O₃ is preferably present in an amount of the additive composition between about 0.5-30.1% weight, Fe₂O₃ is preferably between about 0.5-30.1% weight, Fe₃O₄ is preferably between about 0.5-30.1% weight, and TiO₂ is preferably between about 0-10% weight. If the percentages of these fine oxide particles, except for SiO₂, fall outside each respective range, the dysmenorrhea alleviation effect is mitigated and the malodorous removal effect, and antibacterial effect is mitigated to a lesser degree.

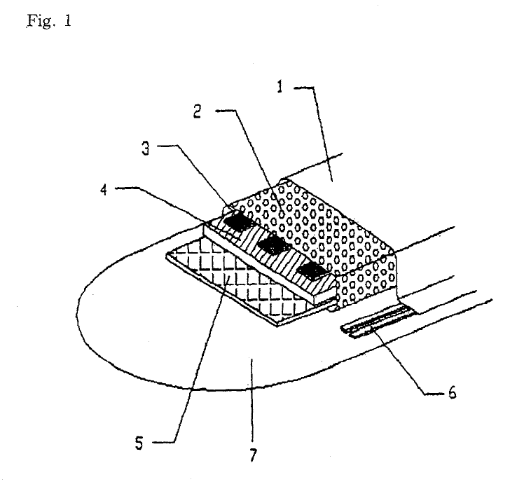

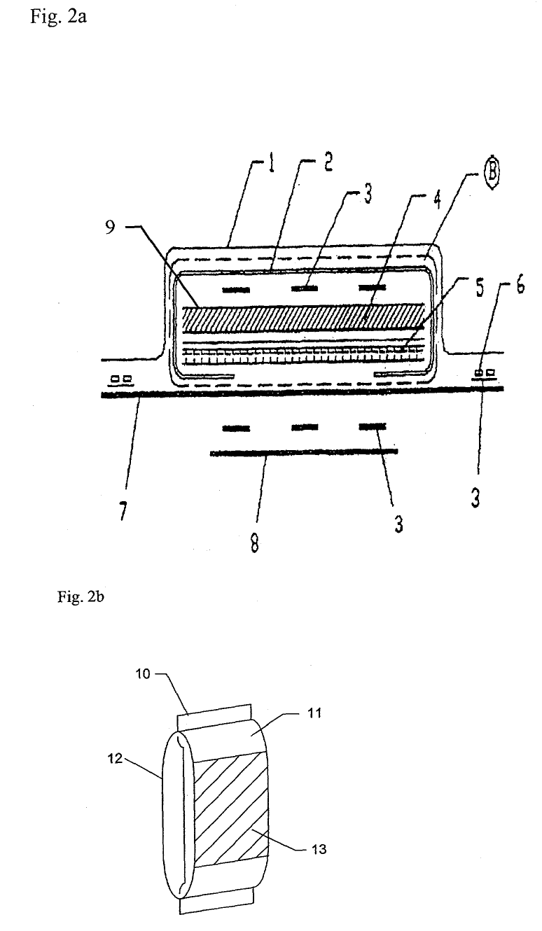

FIG. 1 schematically depicts an exploded perspective view of a general menstrual pad which may contain aspects of the present invention, and FIG. 2 shows a cross-sectional view of the general menstrual pad which may contain aspects of the present invention.

As shown in FIGS. 1 and 2, the menstrual pad preferably includes an absorption surface 1 which may be a dry mesh cover or a perforated film. Absorption surface 1 is preferably liquid permeable and is also preferably non-irritating to a user's skin. It may be manufactured from a variety of conventional materials such as woven and nonwoven polyester, polypropylene, nylon, rayon, and other formed thermoplastic films. Absorbent core B is preferably positioned under the absorption surface 1 for absorbing fluids, e.g., menstrual blood and urine, and backsheet 7 is preferably positioned under absorbent core B for providing support. Backsheet 7 is preferably impervious to liquids and may be made of any number of conventional materials, such as thermoplastic films, e.g., polyethylene.

The absorbent core B, which is preferably positioned between absorption surface 1 and backsheet 7, may be manufactured by any number of fabrication processes depending upon the manufacturer. Within the absorbent core B, pulp fluff 4 may be placed upon an absorption substance sheet 5 or the absorption substance 5 may be scattered upon a center portion of pulp fluff 4. The mixture of fine oxide particles such as SiO₂, Al₂O₃, Fe₂O₃, Fe₃O₄, and TiO₂ is preferably included within pulp fluff 4, as will be described in detail below. The mixture may be placed almost anywhere within the pad so long as it is able to come into contact with the bodily fluids. The amount of the mixture which is effective in relieving pain and mitigating the bacterial growth may range anywhere from 0.2 to 1 gram. Pulp fluff 4 may be made of a number of materials, e.g., fluffed cellulose fibers, wood pulp fibers, blends or masses of fibers, etc., to which the oxide particles may be added.

Absorption substance sheet 5 may be made of materials such as plies of creped cellulose wadding but absorption substance sheet 5 is preferably made of a super absorptive polymer such as polyester or polypropylene, which may be in the form of fibers. Additionally, materials such as corn starch or cellulose may be employed with the super absorption polymer or they may be employed independently of the super absorption polymer.

Absorbent core B may be enveloped by pulp tissue 2 to aid in the absorption of bodily fluids as well as to maintain core B materials positioned relative to one another. The various materials, e.g., pulp fluff 4 and absorption substance 5, are preferably attached to pulp tissue 2 by adhesive agent 3.

Lengths of elastic material 6, which is preferably a lycra-type elastic, may be disposed on either side of absorbent core B and attached to backsheet 7 by adhesive agent 3. Elastic material 6 is preferably covered with absorption surface 1 to aid in conforming the pad to the body of the user as well as helping to retain fluids on absorption surface 1 until they are fully absorbed. Silicon release paper 8 may be disposed along a side of backsheet 7 opposite of absorbent core B to cover adhesive agent 3. Release paper 8 may be removed by the user prior to use such that adhesive agent 3 is exposed. The pad may then be adhered to the undergarment or underwear of the user via adhesive agent 3 for maintaining its position during use.

The inventive construction of the present invention will now be discussed below.

The inventive pad, which may be similarly constructed to conventional pads as described above, further includes the bioceramic substance therein for alleviating multiple conditions, including dysmenorrhea. The inventive pad is preferably used as a menstrual pad, a panty-liner type menstrual pad.

The pad may also be configured for various applications. For instance, a pad having the bioceramic substance may be made into a panty-liner type pad, e.g., one that is extra thin, or it may also be configured into a tampon. Additionally, the pad may simply be configured and used as gauze, e.g., medical gauze. The bioceramic substance may also be directly incorporated into an undergarment, e.g., underwear or panties, by any number of conventional methods, e.g., soaking, spraying, and adhering with adhesive.

Methods of making the inventive pad with the bioceramic substance will be described below. Conventional pads are generally assembled by first positioning pulp fluff 4 onto backsheet 7 or alternatively positioning pulp fluff 4 under absorption surface 1. An absorption substance 5 may be scattered onto pulp fluff 4, or the pulp fluff 4 may be attached onto an absorption substance sheet 5. The absorbent core B may thereby be assembled with pulp fluff 4 and absorption substance sheet 5 to function as an absorption substance, e.g., for absorbing menstrual blood.

The following substances in their respective weight percentages comprising SiO₂ (45-90% weight), Al₂O₃ (0.5-30.1% weight), Fe₂O₃ (0.5-30% weight), Fe₃O₄ (0.5-30.1% weight), and TiO₂ (0-10% weight) are preferably ground into fine particles and any foreign materials which may be harmful to the human body are removed from the substances preferably prior to the oxide or ceramic substances being ground.

Experiments have determined that the degree of the alleviation effects are related to the size of the particles of the various substances. The finer the particle size, the more improved the alleviation effects have been reported. To have a noticeable effect, the particles have diameters preferably on the order of micrometers and are preferably in crystalline form. The various substances described above are preferably inert with no heavy metals present, i.e., no more than trace amounts of heavy metals. The degree of trace amounts may be similarly correlated to standards as recommended by organizations, e.g., the World Health Organization, or the cosmetics industry. Moreover, the various substances preferably have a minimum purity level prior to their being ground and mixed to form the bioceramic mixture. For instance, SiO₂ should ideally have a purity level of at least about 90% before being admixed.

After the substances are ground or otherwise appropriately prepared according to the description herein, they can be implemented by a variety of methods, some examples of which are described below.

The ground ceramic materials may be manufactured by conventional methods into a composite sheet 9 and this sheet 9 may be attached onto pulp fluff 4. Composite sheet 9 is shown in FIG. 2 as being placed atop pulp fluff 4, but sheet 9 may alternatively be placed beneath pulp fluff 4 or anywhere else within the pad. Alternatively, the ground ceramic materials may be scattered onto pulp fluff 4, preferably along a center portion of the pulp fluff 4. Another alternative method is to precompound the ceramic substances and add it to absorption substance 5. After the precompounding process, the ceramic substance may be in a granular, pellet, or powder form.

Aside from the use in a pad, the bioceramic or oxide substance may be encapsulated within a package 10 having semi-permeable walls, such as a tea bag-type package, as seen in FIG. 2b. Such an application would contain the bioceramic substance within the package 10 while allowing fluids to be interchanged through the walls. The package may also have an adhesive 13 on a first side 11; this would allow the package to be adhered to a surface, e.g., an undergarment or panty, while exposing a second side 12 for placement against the skin of a user. After use, the package 10 may then be selectively removable and disposed.

The methodology of the ceramic substance in alleviating dysmenorrhea, removing malodorous scents, and the antibacterial effect will be described below.

A principle of the dysmenorrhea alleviation effect is that various kinds of treatments (e.g., magnetic effects, thermal treatments, resonance effects, regulation of pain transfer substances, and blood circulation improvement) in alleviating uterine ischemia initiate a mutual chain reaction and thereby prevent menstrual cramping within a short period of time.

Also, removal of malodorous scents is initiated by an absorption reaction via ion exchange. It is well known that various bacteria (over 20 kinds) such as a colon bacillus, E. coli, pseudomonas, trichomonas, etc., grow in the genital region of most women. During menstrual periods, bacteria may produce chemicals such as ammonia, trimethylamine, hydrogen sulfide, mercaptane, and amine, that have cationic particles with strong electrical characteristics. The fine oxide particles are negatively charged (i.e., anionic) and form gaps of between about 30-60 μm in diameter between particles. Because the oxide particles are preferably in a powder form, the particles may have a large aggregate surface area. Accordingly, the fine oxide particles may have an overall large surface area per unit volume, which is larger than the apparent geometric area. Not wishing to be bound by theory, the SiO₂ and/or Al₂O₃ preferably contained in the oxide or ceramic material may couple to O₂, thereby generating surface polarity by either a weak Van der Waals' force or dipolar interaction. Since the force induced by the particles may be very strong, absorption phenomenon occurs when the ceramic material is contacted with substances, e.g., menstrual blood or urine or the malodorous gases that may arise therefrom, that are ionically charged.

That is, electrons may move between the oxide surface and any malodorous gases which may arise, thereby generating an interaction between ions via electron sharing which results in absorption of malodorous scents and gases. These malodorous scents and gases may also be alleviated when the electrons are contacted by bodily fluids, such as menstrual blood, because an oxide surface compound may be formed and absorption may also occur when the fluids and gases become fixed in a lattice structure formed by the ceramic materials.

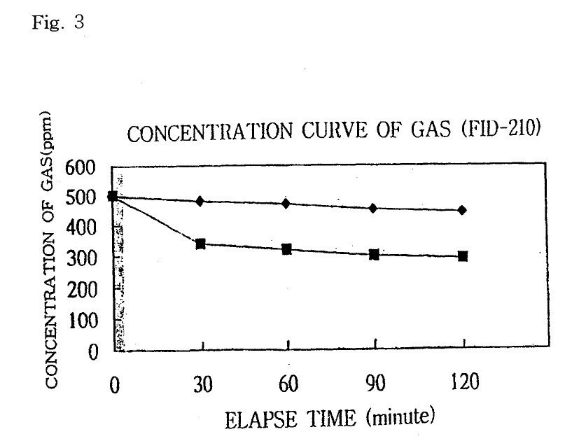

This effect can be seen from Tables 1, 2 and FIG. 3, which illustrates a gas density curve over an elapsed period of time. Table 1 illustrates a negative ion measurement table with the ceramic substances. Table 2 illustrates a deodorization test table with the ceramic substances.

TABLE 1
Negative ion measurement table with the ceramic substances.

| Item sample name | Negative ion (ions/cc) |
|---|---|
| Silica powder | 80 |

1) Test method: KICM-FIR-1042

2) Test piece: Cylinder having an inner diameter=44 mm and a height=297 mm.

3) The test was performed using a charged particle measuring device at room temperature=27° C., humidity=58%, and with the number of negative ions in the atmosphere=77/cc. By measuring the emitted negative ions, the table displays the number of ions per unit volume.

TABLE 2
Deodorization test table with the ceramic substances.

| | | Blank | | |
|---|---|---|---|---|
| | | density | Sample | Deodorization |
| Test Item | Time (min) | (ppm) | density (ppm) | ratio (%) |
| Deodor- | Initial time | 500 | 500 | — |
| ization | 30 | 480 | 340 | 29 |
| | 60 | 470 | 320 | 32 |
| | 90 | 450 | 300 | 33 |
| | 120 | 440 | 290 | 34 |

1) Test method: KICM-FIR-1004

2) Testing gas: Ammonia

3) Gas density measurement: Gas detection tube

4) Test piece: 40×40×10 mm

Where, the reference symbol (−) shown in Table 2 indicates a state having no sample. The results of Table 2 may be seen graphically in FIG. 3. As shown, over an elapsed period of time, the concentration of measured gas decreased significantly indicating a reduction in the emitted odor.

Conventionally, substances such as zeolites have been utilized for filtering-type applications. Generally, zeolites are framework silicates formed of interlocking tetrahedrons of SiO₄ and AlO₄. This alumino-silicate structure is useful in retaining relatively large molecules and cation groups. However, zeolites have been found to be ineffective in alleviating the effects of dysmenorrhea.

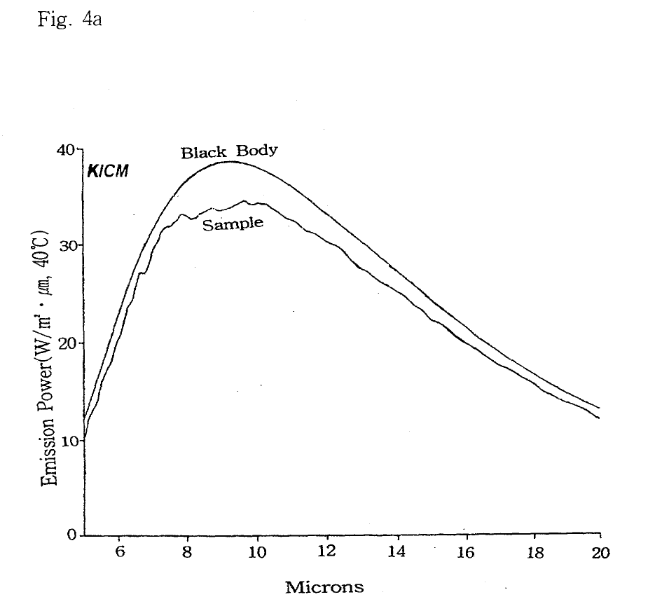

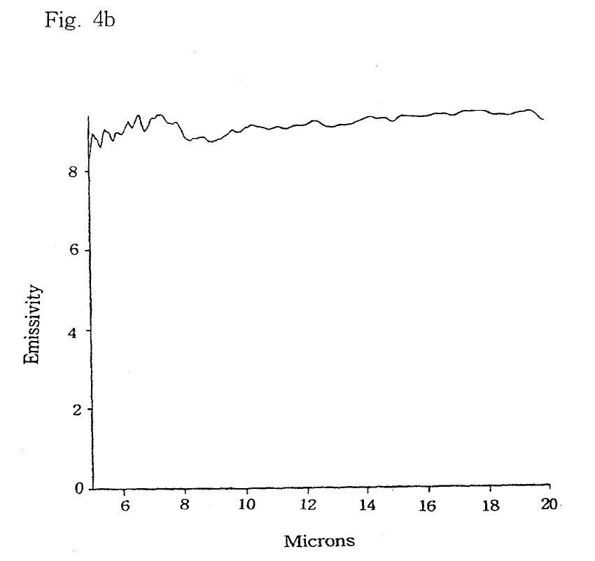

The antibacterial effect may be attributed to a high negative ion concentration and the far infrared rays of the ceramic substance. This may be readily seen from Table 3 and FIGS. 4a and 4b, respectively illustrating the emission energy of far infrared rays and the emissivity of far infrared rays of the ceramic substance.

TABLE 3
Far infrared rays emissivity and an emission energy about
bioceramic substances.

| Emissivity (5-20 μm) | Emission energy (W/m²) |
|---|---|
| 0.911 | 3.67 × 10² |

1) This test was performed at a temperature of 40° C. and is a measurement (using a FT-IR spectrometer) relative to a blackbody.

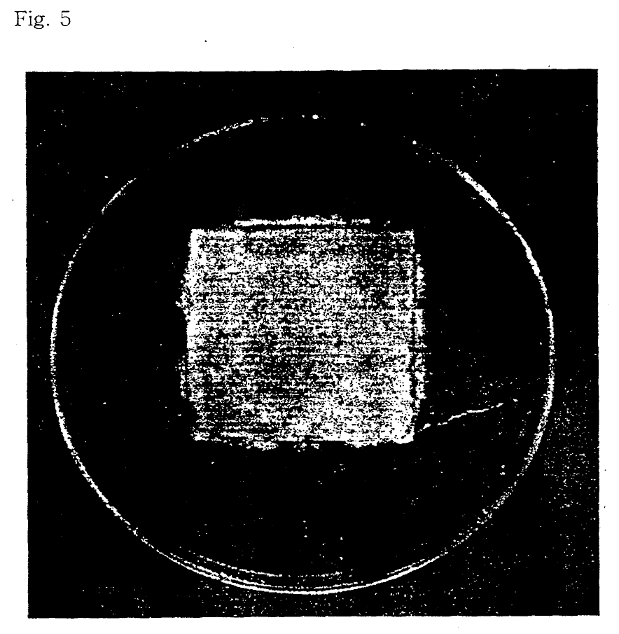

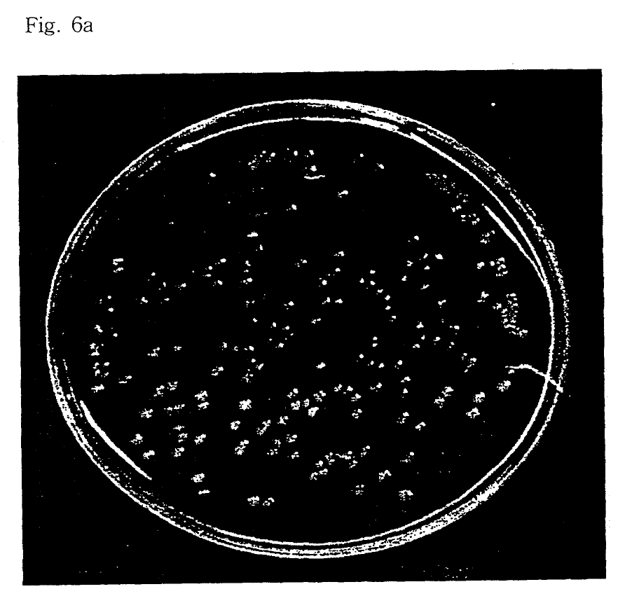

In addition, the antibacterial effect of the ceramic substance can be readily seen from Tables 4 and 5, and FIGS. 5, 6a, 6b, 7a and 7b.

TABLE 4
Anti-fungal testing table with the ceramic substance.

| | | Cultivation test period | | | |
|---|---|---|---|---|---|
| Sample name | Test item | After | After | After | After |
| Silica powder | Anti-fungal test | 1 week | 2 weeks | 3 weeks | 4 weeks |
| | | 2 | 4 | 4 | 4 |

1) Testing method: ASTM G-21

2) Fungal strain (mixture strain):

Aspergillus ATCC 9642

Penicillium pinophilum ATCC 11797

Chaetomium globasum ATCC 6205

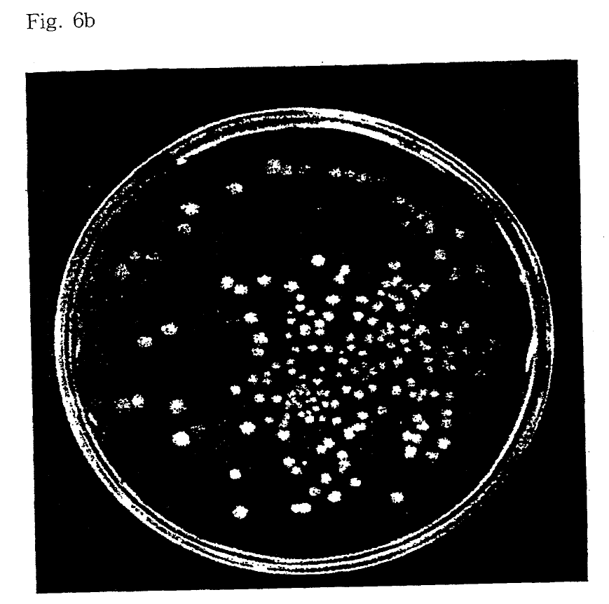

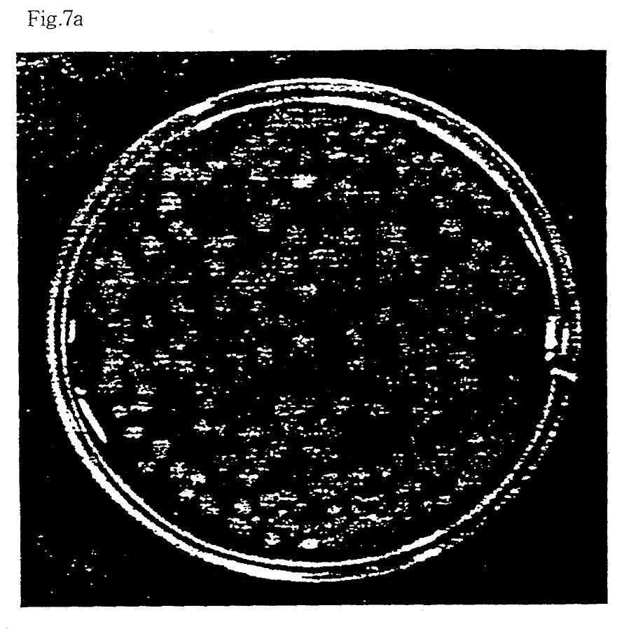

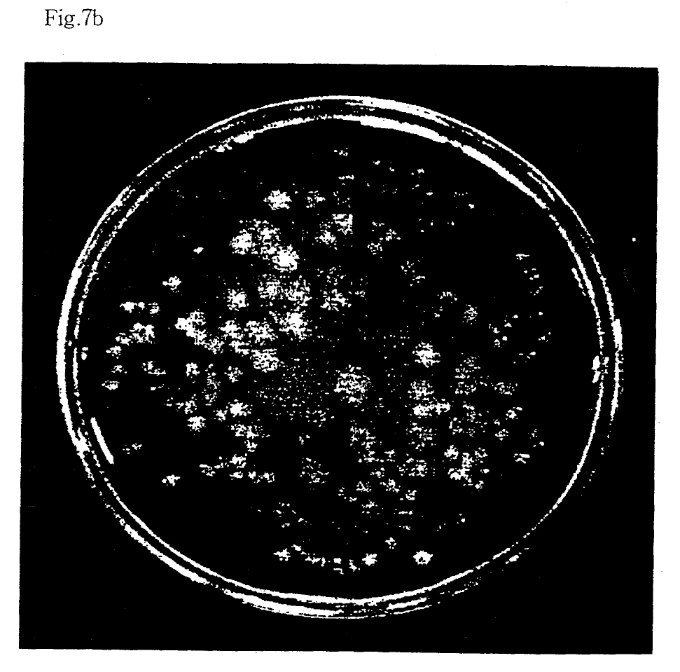

A plurality of molds are grown on each sample surface of FIGS. 6a, 6b and 7a, 7b.

TABLE 5
Antibacterial experiment with the ceramic substance.

| | | Initial | Density after | Bacteria |
|---|---|---|---|---|
| Test | | density | 24 hours | reduction ratio |
| Item | Sample | (CFU/40 p) | (CFU/40 p) | (%) |
| Antibacterial | Blank | 224 | 566 | — |
| experiment with | Silica | 224 | 218 | 2.7 |
| E. coli | powder | | | |
| Antibacterial | Blank | 255 | 706 | — |
| experiment with | Silica | 255 | 244 | 4.3 |
| Pseudomonas | powder | | | |

1) Test method: KICM-FIR-1002

2) Strain used:

Escherichia coli ATCC 25922

Pseudomonas aeruginosa ATCC 15442

In Table 5, CFU is the colony forming unit, 40 p is 0.04 ml, and Blank is the measurement result where no sample of the ceramic material was introduced, i.e., where the bacteria was allowed to grow uninhibited. As seen, the density of the bacteria in the Blank sample grew substantially over a 24 hour period, yet the density actually decreased in both samples of bacteria where silica powder was introduced.

Using prototype samples, the following experimental clinical trials shown in Table 6 were performed giving favorable results.

TABLE 6
Experimental clinical trials.

| Test name | Doctor & details | Duration |
|---|---|---|
| Primary clinical | Seong-Lim KIM | 09/01/99-12/31/99 |
| demonstration | | |
| Material test | Component analysis | 07/01/00-09/30/00 |
| | Harmful material elution test | |
| | Deodorization test | |
| | Antibacterial (E. coli, | |
| | pseudomonas) test | |
| | etc | |
| Secondary clinical | Professor Tae-Sung LEE | 10/01/00-4/01 |
| demonstration | Professor Moo-Sik LEE | |
| | Professor Chung-Won LEE | |

Primary Clinical Experiments

Primary clinical demonstrations were performed to verify the dysmenorrhea alleviation effects attributed to the inventive pad.

The bioceramic or oxidic substance was finely ground into particle sizes no larger than 150 mesh (104 μm); it is more preferable that the particles have a size no larger than 300 mesh (46 μm). The composition of the mixture included SiO₂ (90% weight), Al₂O₃ (5-10% weight), Fe₃O₄ (1% weight), and TiO₂ (1% weight).

A. Test I

Sixty women of menstruation age were selected at random; all of the women were selected from a group which had a habit of taking anodynes (i.e., analgesics) during menstruation. The dysmenorrhea alleviation effect provided by the inventive menstrual pad was fully explained to these women. Then, the sixty women were divided into four groups for more effective testing results.

TABLE 7
Clinical demonstration results (number experiencing dysmenorrhea
alleviation/pain continuation).

| | A | B | C | D (10 | |
|---|---|---|---|---|---|
| Month | (10 women) | (10 women) | (10 women) | women) | Totals |
| 1 | 10/0 | 17/3 | 20/0 | 10/0 | 57/3 |
| 2 | 10/0 | 18/2 | 20/0 | 10/0 | 58/2 |

Where, the A group was composed of women in their teens (teenagers), B group of women in their 20's, C group of women in their 30's, and D group of women in their 40's.

Observations:

1) Three women out of 60 women reported a rise in their body temperature in the vicinity of their lower abdomen, but they continuously felt menstrual cramp pain.

2) After using the inventive menstrual pad, most women (except for the three) reported feeling alleviation from dysmenorrhea.

B. Test II

In order to observe any placebo effects, twenty women (except for the three mentioned above) who continuously felt menstrual cramp pain were selected at random during the time period of November-December, 1999. Accordingly, the test operator told them a falsehood that they were wearing the inventive menstrual pad and not a conventional menstrual pad.

TABLE 8
Clinical demonstration result (number experiencing dysmenorrhea
alleviation/pain continuation).

| | A (5 | B (15 | C (15 | D (5 | Total (40 | Placebo (20 |
|---|---|---|---|---|---|---|
| Month | women) | women) | women) | women) | women) | women) |
| 3 | 5/0 | 13/2 | 15/0 | 5/0 | 38/2 | 5/15 |
| 4 | 5/0 | 13/2 | 14/1 | 5/0 | 37/3 | 4/16 |

Where, the A group was composed of women in their teens (teenagers), B group of women in their 20's, C group of women in their 30's, and D group of women in their 40's.

Observations:

1) Most of the 20 women (about 77.5%) undergoing the placebo effect still complained of menstrual cramp pain.

2) Two women continuously complained of menstrual cramp pain were exhorted by the test operator to receive medical treatment by a gynecologist.

C. Test III

Based on the above tests I and II, the test results indicate that the inventive menstrual pad can alleviate dysmenorrhea. Twenty more women were selected to compare the pain alleviation effects of the inventive pad against the effects of taking an anodyne. The comparison result has been expressed as a score ranging from 1 to 100.

TABLE 9
Clinical demonstration result.

| | A | B | C | D | |
|---|---|---|---|---|---|
| Month | (5 women) | (5 women) | (5 women) | (5 women) | |
| 5 | 70 | 85 | 90 | 92 | |
| | 85 | 87 | 93 | 95 | |
| | 90 | 90 | 95 | 95 | |
| | 85 | 95 | 97 | 96 | |
| 6 | 72 | 84 | 91 | 92 | |
| | 85 | 87 | 93 | 95 | |
| | 91 | 90 | 95 | 95 | |
| | 88 | 95 | 97 | 96 | |
| Average | 85 | 90.3 | 94.6 | 94.8 | 91.1 |

Where, the A group was composed of women in their teens (teenagers), B group of women in their 20's, C group of women in their 30's, and D group of women in their 40's. A score over 90=very satisfactory, a score over 80=satisfactory, a score over 70=good, and a score of 60=average.

Observations:

1) Most women reported a high satisfaction state with an average score of 91.

2) Women in their 30's and 40's felt a higher satisfaction state than women in their teens and 20's.

As described above, the multi-functional absorption pad comprising ceramic substances therein or thereon, according to the present invention, may simultaneously alleviate dysmenorrhea, malodorous scents, and bacterial growth, thereby ensuring a woman's normal social activity even in her state of dysmenorrhea.

That is, menstrual cramp pains may be remarkably alleviated, unpleasant odors may be reduced, and feelings of a cold lower abdomen and unpleasant feelings in the lower abdomen may also be remarkably reduced.

It is understood that various other modifications will be apparent and can be readily made by those skilled in the art without departing from the scope and spirit of the invention. Accordingly, it is not intended that the scope of the claims appended hereto be limited to the description as set forth herein, but rather that the claims be construed as encompassing all the features of patentable novelty that reside in the present invention, including all features that would be treated as equivalents thereof by those skilled in the art to which this invention pertains.

We claim:

1. A multi-functional pad for absorbing bodily fluids, comprising:
an absorption surface;
an absorbent core for absorbing the bodily fluids, wherein the core is positioned adjacent to the absorption surface; and
a backsheet layered adjacent to the absorbent core opposite the absorption surface,
wherein the absorbent core comprises a ceramic or oxidic substance or additive dispersed within the core for contacting the bodily fluids.

2. The pad of claim 1 wherein the absorbent core further comprises a layer of pulp fluff disposed therewithin.

3. The pad of claim 2 wherein the ceramic substance is disposed within or upon the layer of the pulp fluff.

4. The pad of claim 1 wherein the absorbent core further comprises a layer of an absorption substance disposed therewithin.

5. The pad of claim 1 wherein the ceramic or oxidic substance or additive comprises oxide particles.

6. The pad of claim 5 wherein the ceramic or oxidic substance or additive further comprises SiO₂.

7. The pad of claim 6 wherein the SiO₂ comprises 45-90% weight of the ceramic or oxidic substance or additive.

8. The pad of claim 6 wherein the ceramic or oxidic substance or additive further comprises Al₂O₃, Fe₂O₃, and Fe₃O₄.

9. The pad of claim 8 wherein Al₂O₃, Fe₂O₃, and Fe₃O₄ each comprise 0.5-30.1% weight of the ceramic or oxidic substance or additive.

10. The pad of claim 8 wherein the ceramic or oxidic substance or additive further comprises TiO₂ comprising 0-10% weight of the ceramic or oxidic substance or additive.

11. The pad of claim 1 wherein the pad comprises a menstrual pad, a panty-liner type pad, or a tampon.

12. The pad of claim 1 wherein the ceramic or oxidic substance or additive is in a form selected from the group consisting of powders, grains, and pellets.

13. The pad of claim 1 wherein the ceramic or oxidic substance or additive is negatively charged.

14. The pad of claim 1 wherein the ceramic or oxidic substance or additive is in a powder form, the powder comprising particles no larger than 104 μm.

15. A multi-functional absorption pad for absorbing menstrual fluids, comprising:
an absorption surface;
an absorbent core for absorbing the bodily fluids comprising an absorption substance sheet and a layer of pulp fluff disposed under the absorption substance sheet, wherein the core is positioned adjacent to the absorption surface; and
a backsheet layered adjacent to the absorbent core opposite the absorption surface,
wherein the absorbent core comprises a ceramic or oxidic substance or additive dispersed within the pulp fluff, the ceramic or oxidic substance or additive comprising 45-90% weight of SiO₂, 0.5-30.1% weight of Al₂O₃, 0.5-30.1% weight of Fe₂O₃, 0.5-30.1% weight of Fe₃O₄, and 0-10% weight of TiO₂.

16. A biocompatible ceramic or oxidic mixture for use in alleviating pain comprising:
a mixture of oxide particles comprising SiO₂, Al₂O₃, Fe₂O₃, Fe₃O₄, and TiO₂,
wherein the SiO₂ comprises 45-90% weight of the ceramic or oxidic mixture,
wherein the Al₂O₃, Fe₂O₃, and Fe₃O₄ each comprise 0.5-30.1% weight of the ceramic mixture, and
wherein the TiO₂ comprises 0-10% weight of the ceramic or oxidic mixture.

17. The ceramic or oxidic mixture of claim 16 wherein the mixture is incorporated into a pad adapted to absorb bodily fluids.

18. The ceramic or oxidic mixture of claim 17 wherein the pad comprises a type selected from the group consisting of menstrual pads, panty-liner type pads, tampons, and gauze.

19. The ceramic or oxidic mixture of claim 16 wherein the mixture is directly incorporated into an undergarment.

20. The ceramic or oxidic mixture of claim 19 wherein the mixture is incorporated into the undergarment by a method selected from the group consisting of soaking, spraying, and adhering.

21. The ceramic or oxidic mixture of claim 16 wherein the mixture is incorporated into a package having semi-permeable walls adapted to contain the mixture within the package while permitting a fluid to pass through the walls.

22. The ceramic or oxidic mixture of claim 21 wherein the package comprises a tea bag-type package.

23. The ceramic or oxidic mixture of claim 21 wherein the package comprises a first side and a second side, the package further comprising an adhesive attached to the first side for selectively adhering to a surface.

24. The ceramic or oxidic mixture of claim 16 wherein the ceramic mixture is in a form selected from the group consisting of powders, grains, and pellets.

25. The ceramic or oxidic mixture of claim 16 wherein the ceramic or oxidic mixture is negatively charged.

26. The ceramic or oxidic mixture of claim 16 wherein the ceramic or oxidic substance is in a powder form, the powder comprising particles no larger than 46 μm.

27. The ceramic or oxidic mixture of claim 16 wherein the SiO₂ comprises 90% weight of the ceramic or oxidic mixture.

28. The ceramic or oxidic mixture of claim 16 wherein the Al₂O₃ comprises 5-10% weight of the ceramic or oxidic mixture.

29. The ceramic or oxidic mixture of claim 16 wherein the Fe₃O₄ comprises 1% weight of the ceramic or oxidic mixture.

30. The ceramic or oxidic mixture of claim 16 wherein the TiO₂ comprises 1% weight of the ceramic or oxidic mixture.

31. The ceramic or oxidic mixture of claim 16 wherein the total weight of the ceramic or oxidic mixture is between 0.2-1 gram.

32. A method of manufacturing a multi-functional absorption pad for absorbing bodily fluids, comprising:
processing a ceramic or oxidic substance into particles, the ceramic or oxidic substance comprising a clay mineral and SiO₂; and
combining the ceramic or oxidic substance with an absorbent core material for placement within the pad.

33. The method of claim 32 wherein the ceramic or oxidic substance further comprises Al₂O₃, Fe₂O₃, Fe₃O₄, and TiO₂.

34. The method of claim 32 wherein the ceramic or oxidic substance comprises 45-90% weight of SiO₂.

35. The method of claim 33 wherein the ceramic or oxidic substance comprises 0.5-30.1% weight of each of Al₂O₃, Fe₂O₃, Fe₃O₄, and 0-10% weight of TiO₂.

36. The method of claim 32 wherein processing the ceramic or oxidic substance into particles comprises forming the ceramic substance into a form selected from the group consisting of grains, pellets, and powders.

37. The method of claim 32 wherein combining the ceramic or oxidic substance with the absorbent core material comprises scattering the ceramic or oxidic substance over the absorbent core material.

38. The method of claim 32 wherein combining the ceramic or oxidic substance with the absorbent core material comprises forming the ceramic or oxidic substance into a sheet for placement upon the absorbent core material.
